(19) United States
(12) Patent Application Publication
(10) Pub. No.: US 2010/0158980 A1
(43) Pub. Date: Jun. 24, 2010

(54) DRUG DELIVERY DEVICES FOR DELIVERY OF THERAPEUTIC AGENTS

(76) Inventors: Casey Kopczynski, Chapel Hill, NC (US); Cheng-Wen Lin, Durham, NC (US); Chris Sutay, Raleigh, NC (US)

Correspondence Address: MICHAEL BEST & FRIEDRICH LLP, 100 E WISCONSIN AVENUE, Suite 3300, MILWAUKEE, WI, 53202, US

(21) Appl. No.: 12/337,898

(22) Filed: Dec. 18, 2008

(51) Int. Cl. A61F 2/00

(52) U.S. Cl. 424/426

(57) ABSTRACT

Drug delivery devices comprising a non-bioabsorbable polymer structure and a composition comprising an active agent have been discovered. The drug delivery devices may be used to treat ocular conditions, among other diseases and conditions. In addition, a method of treating an ocular condition has been discovered comprising implanting a drug delivery device which releases the active agent at a rate of

Q=0.001×N×C

wherein C is the topical effective concentration (in milligram/mL) of the active agent and N=0.01 to 0.5 for prostaglandins in their ester, amide, free acid or salt form, and N=0.5 to 5 for any active agent other than prostaglandins in their ester, amide, free acid or salt form.

### FIELD OF THE INVENTION

The present invention relates to an implantable drug delivery device for sustained delivery of therapeutic agents. In particular, it relates to a non-biodegradable, drug-eluting removable device for tissue implantation for the purpose of treating various diseases and conditions. More particularly, but not by way of limitation, this device is well-suited for episcleral implantation and delivery of pharmaceutical agents for the treatment of glaucoma and ocular hypertension.

### BACKGROUND

The delivery of therapeutic and pharmaceutical agents is a complex problem without a single universal solution. Many chronic diseases and conditions can be treated effectively by oral medications, but side effects, patient forgetfulness, and other factors often produce high rates of noncompliance with the recommended treatment. In such cases, patient outcomes can be improved using sustained delivery formulations that simplify the medication regimen (e.g., Lupron Depot® for endometriosis).

Where possible, diseases and conditions that affect only a single organ or local tissue are preferably treated by a local application. This allows for a relatively high concentration of the therapeutic agent at the site where it is most needed, and allows for minimal systemic exposure. However there are relatively few tissues that are directly accessible, with skin, hair follicles, the oral, nasal and genitourinary cavities, and eyes being candidates for direct application of therapeutic agents. Direct application of therapeutic agents to internal organs is more challenging, but has been useful in the treatment of some types of tumors.

In the treatment of ocular conditions in particular, many medications are now delivered topically to the eye as eyedrops. Despite the success of the eyedrop in treating diseases and conditions of the eye, treatment with topical eyedrops suffers from numerous drawbacks.

A significant drawback of the eyedrop is the requirement that the pharmaceutical agent be soluble in an isotonic buffered solution at a therapeutically effective concentration and be chemically stable in solution for 18 months or longer. However, solubility of useful therapeutic agents in aqueous formulation is often well below the concentration needed for effective treatment. This can sometimes be corrected by the addition of various excipients, but this increases the complexity of the formulation and often reduces tolerability of the eyedrop.

A second limitation of eyedrops is the rapid clearance of the therapeutic agent via nasolacrimal drainage from the eye surface. This results in most of the compound being delivered to the inside of the nose, where it is not needed and where, in fact, a high concentration of agent might have a detrimental effect.

A third limitation to the use of eyedrops is the observation that many therapeutically-valuable agents cause a local irritation when topically-dosed to the eye. The cornea of the eye is highly sensitive to the application of chemical agents. This irritation potential significantly limits the use of many otherwise valuable therapeutic agents.

A fourth limitation of eyedrops, which also applies to systemic drugs taken by oral, sublingual, nasal or rectal delivery routes, is the need to re-apply the therapeutic agent on a regular basis. For eyedrops, repeating application as frequently as four times a day can be necessary, and even the best agents must be reapplied on a daily basis. For many individuals, in particular the elderly, this frequent dosing becomes burdensome and leads to non-compliance with the dosing regimen, lessening the therapeutic value of the treatment.

To counter these disadvantages of eyedrop delivery, researchers have suggested various devices aimed at providing local delivery over a longer period of time. U.S. Pat. No. 5,824,072 to Wong discloses a non-biodegradable implant containing a pharmaceutical agent that diffuses through a water-impermeable polymer matrix into the target tissue. The implant is placed in the tear film or in a surgically-induced avascular region, or in direct communication with the vitreous.

U.S. Pat. No. 5,476,511 to Gwon et al. discloses a polymer implant for placement under the conjunctiva of the eye. The implant is claimed to be useful for the delivery of neovascular inhibitors for the treatment of age-related macular degeneration (AMD). Again, the pharmaceutical agent diffuses through a water-impermeable polymer matrix of the implant.

U.S. Pat. No. 5,773,019 to Aston et al. discloses a non-biodegradable implant for the delivery of steroids and immunosuppressives such as cyclosporine for the treatment of uveitis, with the drug again diffusing through the water-impermeable polymer matrix of the implant.

U.S. Pat. No. 3,854,480 to Zaffaroni discloses a drug-delivery system with a solid inner matrix formulation containing solid particles of drug surrounded by an outer polymer membrane that is permeable to the passage of the drug. While both the inner matrix and the outer wall are claimed to be permeable to the passage of drugs, the patent requires that the rate of diffusion of the outer membrane be not more than 10% of the rate of the inner matrix.

Both U.S. Pat. No. 4,281,654 to Shell, et al. and U.S. Pat. No. 4,190,642 to Gale, et al. disclose matrix polymer systems that are designed to deliver either beta-blockers or a combination of epinephrine and pilocarpine to the eye to treat glaucoma. Gale, et al. micronize their medicaments to a particle size of not more than 100 microns and these are subsequently dispersed throughout the entire polymer matrix, with no distinct cavity that contains the drug and no drug-free outer layer. In addition, both Shell and Gale require the walls surrounding these small depots be ruptured by the force of the osmotic pressure in order to release the drug by way of those formed ruptures.

All of the above-referenced patents and publications are hereby incorporated herein by reference.

### SUMMARY OF THE INVENTION

In one aspect, the present invention may provide a drug delivery device having a non-bioabsorbable polymer structure enclosing a composition comprising an active agent, wherein the polymer structure comprises a mixture comprising a water-soluble polymer and a non-water-soluble polymer.

In another aspect, the present invention may also provide a drug delivery device having a non-bioabsorbable polymer structure enclosing a composition comprising an active agent, wherein the polymer structure comprises an impermeable polymer through which the active agent does not permeate and a partially-bioerodible membrane through which the active agent permeates.

In yet a further aspect, the present invention may provide a drug delivery device having a non-bioabsorbable polymer structure enclosing a composition comprising a single compressed pellet comprising an active agent with a solubility of greater than about 50 micrograms/mL in phosphate buffered saline at neutral pH, wherein the polymer structure comprises an impermeable polymer through which the active agent does not permeate and a rate-limiting water-permeable polymer through which the active agent permeates.

In yet another aspect, the present invention may provide a drug delivery device having a composition comprising an active agent at least partially encompassed by an impermeable membrane and a permeable membrane, wherein the permeable membrane controlling release of the active agent episclerally over a period of time.

In a further aspect, the present invention may provide methods of using the drug delivery devices to treat ocular conditions, among other diseases and conditions. In some embodiments, the drug delivery device is implanted at or near a tissue affected by the ocular condition.

In another aspect, the present invention may provide a method of treating an ocular condition comprising implanting episclerally a drug delivery device comprising an active agent, wherein the active agent is released at a rate of

Q=0.001×N×C

wherein C is the topically effective concentration (in milligram/mL) of the active agent and N=0.01 to 0.5 for prostaglandins in their ester, amide, free acid or salt form, and N=0.5 to 5 for any active agent other than prostaglandins in their ester, amide, free acid or salt form.

### BRIEF DESCRIPTION OF THE DRAWINGS

FIG. 1 shows a drug delivery device according to the present invention.

FIG. 2 shows a drug delivery device according to the present invention.

FIG. 3 shows the release profile for a drug delivery device according to the present invention.

FIG. 4 shows the release profile for a drug delivery device according to the present invention.

FIG. 5 shows the IOP-lowering effect of a drug delivery device according to the present invention.

FIG. 6 shows the release profile of a drug delivery device according to the present invention.

FIG. 7 shows the IOP-lowering effect of a drug delivery device according to the present invention.

FIG. 8 shows the release profile of a drug delivery device according to the present invention.

FIG. 9 shows the IOP-lowering effect of a drug delivery device according to the present invention.

FIG. 10 shows the release profile of a drug delivery device according to the present invention.

FIG. 11 shows the release profile of a drug delivery device according to the present invention.

FIG. 12 shows the release profile of a drug delivery device according to the present invention.

FIG. 13 shows the IOP-lowering effect of a drug delivery device according to the present invention.

FIG. 14 shows the release profile of a drug delivery device according to the present invention.

FIG. 15 shows the release profile of a drug delivery device according to the present invention.

FIG. 16 shows the IOP-lowering effect of a drug delivery device according to the present invention.

FIG. 17 shows a flowchart for designing drug delivery devices.

FIG. 18 shows a flowchart for designing drug delivery devices.

FIG. 19 shows solubility characteristics for various active agents.

### DETAILED DESCRIPTION

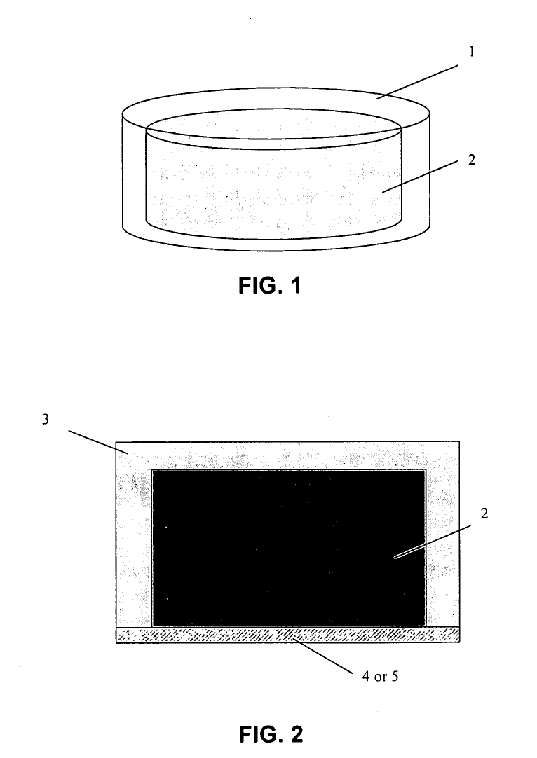

The drug delivery devices of the present invention comprise a non-bioabsorbable polymer structure which encloses a composition comprising an active agent (2, FIGS. 1 and 2). The active agent is released through the polymer structure once the drug delivery device is implanted in the desired portion of the body.

The non-bioabsorbable polymer structure comprises, in one embodiment shown in FIG. 1, a mixture (1) comprising a water-soluble polymer and a non-water soluble polymer with about 0% to about 50% by weight of the mixture being the water-soluble polymer or about 10% to about 30% by weight. Suitably, the drug delivery device at least partially bioerodes when implanted in the body as the water-soluble polymer dissolves leaving a porous non-bioabsorbable polymer structure through which the active agent is released. The polymer structure suitably has a thickness of about 20 micrometers to about 800 micrometers or about 40 micrometers to about 500 micrometers or about 50 micrometers to about 250 micrometers, depending on the overall size and required mechanical strength of the device.

The non-water soluble polymer may be selected from ethylene vinyl acetate (EVA), silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutymethacrylate, plasticized polyethyleneterephthalate, polyisoprene, polyisobutylene, silicon-carbon copolymers, natural rubber, plasticized soft nylon, polytetrafluoroethylene (PTFE), or combinations thereof. Suitably, the non-water soluble polymer is EVA. The vinyl acetate content may be from about 9% to about 50% by weight (EVA-9-50). In one embodiment, the vinyl acetate content is about 40% by weight (EVA-40). Other suitable non-water soluble polymers are known to those of ordinary skill in the art.

The water-soluble polymer may be selected from dextran, cyclodextrin, poly-(L-lactic acid), polycaprolactone, poly(lactic-co-glycolic acid), poly(glycolic acid), poly(trimethylene carbonate), polydioxanone or combinations thereof. Other suitable water-soluble polymers are known to those of ordinary skill in the art.

Alternatively, in an embodiment shown in FIG. 2, the non-bioabsorbable polymer structure comprises an impermeable polymer (3) and a partially-bioerodible membrane (4). Suitably, about 0% to about 50% by weight of the polymer structure is the partially-bioerodible membrane or about 10% to about 30% by weight of the partially-bioerodible membrane. The impermeable polymer does not allow the passage of the active agent and provides mechanical strength for the device. The impermeable polymer suitably has a thickness of about 50 micrometers to about 800 micrometers or about 100 micrometers to about 250 micrometers, depending on the overall size and required mechanical strength of the device. The partially-bioerodible membrane suitably has a thickness of about 20 micrometers to about 800 micrometers or about 40 micrometers to about 500 micrometers, depending on the overall size and required mechanical strength of the device.

Suitable impermeable polymers include, but are not limited to, EVA-9-50, silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutylmethacrylate, plasticized polyethyleneterephthalate, polyisoprene, polyisobutylene, silicon-carbon copolymers, natural rubber, plasticized soft nylon, polytetrafluoroethylene (PTFE), or combinations thereof. Other suitable impermeable polymers are known to those of ordinary skill in the art.

In some embodiments, the partially-bioerodible membrane comprises an impermeable polymer and a bioerodible polymer. Suitably, the partially-bioerodible membrane contains about 0% to about 50% by weight of the bioerodible polymer. Suitable bioerodible polymers include, but are not limited to, dextran, cyclodextrin, poly-(L-lactic acid), polycaprolactone, poly(lactic-co-glycolic acid), poly(glycolic acid), poly(trimethylene carbonate), polydioxanone, or combinations thereof. Other suitable bioerodible polymers are known to those of ordinary skill in the art.

In another embodiment also encompassed by FIG. 2, the non-bioabsorbable polymer structure comprises an impermeable polymer (3) and a rate-limiting water-permeable polymer (5). Suitably, the polymer structure contains about 0% to about 50% by weight of the rate-limiting water permeable polymer or about 10% to about 30% by weight of the rate-limiting water permeable polymer. The impermeable polymer does not allow the passage of the active agent and provides mechanical strength for the device. The impermeable polymer suitably has a thickness of about 50 micrometers to about 800 micrometers or about 100 micrometers to about 250 micrometers, depending on the overall size and required mechanical strength of the device.

Suitable impermeable polymers include, but are not limited to, EVA-9-50, silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutylmethacrylate, plasticized polyethyleneterephthalate, polyisoprene, polyisobutylene, silicon-carbon copolymers, natural rubber, plasticized soft nylon, polytetrafluoroethylene (PTFE), or combinations thereof. Other suitable impermeable polymers are known to those of ordinary skill in the art.

The rate-limiting water-permeable polymer is a polymer that allows for the passage of active agent and water or tissue fluids. The composition and/or thickness of this polymer determines the rate of release from the drug delivery device. The water-permeable polymer has limited water permeability which only allows water passage into the drug core (2) at a very slow rate. Once water penetrates the polymer into the enclosed drug core (2), it then serves as a solvent to dissolve the active agent to its solubility limit. Therefore, the active agent suitably has low or moderate solubility. In one embodiment, the majority of the active agent remains as a solid compressed form and the concentration of the dissolved aqueous portion remains at its solubility limit, so that the concentration gradient across the polymer remains substantially constant, given that the clearance rate is sufficient in the environment. Without wishing to be bound by theory, in one embodiment the above described mechanisms allow this polymer to provide the rate-limiting steps that allow the active agent to be released at a substantially constant rate until at least about 70% to at most about 95% of the active agent is released from the drug delivery device. The rate-limiting water-permeable polymer suitably has a thickness of about 20 micrometers to about 500 micrometers, depending on the overall size and required mechanical strength of the device.

Suitable rate-limiting water-permeable polymers may be selected from ethylene vinyl acetate with a vinyl acetate content of about 26% to about 80% by weight (EVA-26-80) or ethylene vinyl alcohol with a vinyl alcohol content of about 40% to about 80% by weight (EVOH-40-80). Suitable rate-limiting water-permeable polymers may be copolymers that have both hydrophobic and hydrophilic monomers where the hydrophilic portion allows the passage of water or tissue fluids and the hydrophobic portion limits its water-permeability in order to provide the rate-limiting barrier. Other suitable rate-limiting water-permeable polymers are known to those of ordinary skill in the art.

In some embodiments, the drug delivery device has a cylindrical structure. Suitably, the cylindrical structure comprises a cylindrical wall, a top and a bottom. The top and the bottom are coupled to opposite sides of the cylindrical wall. In some embodiments, the cylindrical wall and top comprise the impermeable polymer and the bottom comprises the partially-bioerodible membrane or rate-limiting water-permeable polymer. In other embodiments, drug delivery device can be spherical, tubular, rod-shaped, or the like.

In some embodiments, the non-bioabsorbable polymer structure contains a pigment. The pigment is optionally placed into the impermeable polymer. Suitable pigments include, but are not limited to, inorganic pigments, organic lake pigments, pearlescent pigments, fluorescein, and mixtures thereof. Inorganic pigments useful in this invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this invention include those selected from the group consisting of D&C Red No. 19 (CI 45,170), D&C Red No.9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

In a further embodiment, the drug delivery device comprises a composition comprising an active agent at least partially encompassed by an impermeable membrane and a permeable membrane, wherein the permeable membrane controls release of the active agent episclerally over time.

About 70% to about 90% of the active agent is suitably released from the drug delivery device over a period of about 30 days to about 5 years. Alternatively, about 70% to about 90% of the active agent is released over a period of about 30 days to about 2 years or about 30 days to about 1 year or about 30 days to about 90 days or about 1 year to about 5 years or about 1 year to about 2 years.

In some embodiments, the active agent is released from the drug delivery device at a rate of about 0.0001 micrograms/hr to about 30 micrograms/hr, or from about 0.001 micrograms/hr to about 30 micrograms/hr, or from about 0.001 micrograms/hr to about 10 micrograms/hr.

Suitably, the rate of release of the active agent does not deviate substantially from linearity (i.e., does not deviate from linearity more than about 5%) until at least about 70% and at most about 95% of the active agent is released from the drug delivery device.

Alternatively, about 2% to about 90% of the active agent is released from the drug delivery device with a coefficient of determination, R-squared or R², of the linear regression is at least about 0.95.

Dosages may be varied based on the active agent being used, the patient being treated, the condition being treated, the severity of the condition being treated, the route of administration, etc. to achieve the desired effect.

The drug delivery devices of the present invention can be used to treat various conditions including, ocular conditions (such as glaucoma, ocular hypertension, ocular inflammation, uveitis, macular degenerative conditions, retinal degenerative conditions, ocular tumors, ocular allergy, and dry eye), topical fungal infections, topical bacterial infections, dermatitis, peripheral neuropathy, allergic and other rashes, and topical eruptions of t-cell lymphoma. Some of the drug delivery devices of the present invention are also useful in decreasing intraocular pressure. In addition to treatment of ocular conditions, the present invention can be used for local delivery of therapeutics to various types of solid tumors, including tumors of the lung, pancreas, liver, kidney, colon and brain.

The device can also be implanted subcutaneously, intramuscularly or intraperitoneally for systemic delivery of therapeutics, including delivery of contraceptive agents and agents to treat cardiovascular, metabolic, immunological and neurological disorders. The drug delivery device may be implanted at or near a tissue affected by the condition. The drug delivery devices of the present invention are suitably implanted in ocular tissues. In some embodiments, the drug delivery devices are implanted episclerally (inserted between the conjunctiva and sclera) with the permeable portion of the polymer structure facing the sclera.

In some embodiments, the present invention is a method of treating an ocular condition comprising implanting episclerally a drug delivery device containing a composition comprising an active agent, wherein the active agent is released at a rate of

Q=0.001×N×C

wherein C is the topically effective concentration (in milligrams/mL) of the active agent and N=0.01 to 0.5 for prostaglandins in their ester, amide, free acid or salt form, and N=0.5 to 5 for any active agents other than prostaglandins in their ester, amide, free acid or salt form. Using the equation, a non-prostaglandin active agent with a topical effective concentration of 1.5 milligrams/mL (e.g., a brimonidine salt) or 5 milligrams/mL (e.g., a timolol salt) may be designed to release at a rate of 0.75 to 7.5 micrograms/hr or 2.5 to 20 micrograms/hr, respectively. Using a similar approach, a prostaglandin active agent with a topical effective concentration of 0.05 milligrams/mL (e.g., latanoprost) or 0.04 milligrams/mL (e.g., travoprost) may be designed to release at a rate of 0.0005 to 0.025 micrograms/mL or 0.0004 to 0.02 micrograms/mL, respectively.

The active agent may be any active agent suitable to treat the desired condition. In various embodiments, the active agent may be of one of low solubility, moderate solubility or high solubility. “Low solubility” means a solubility of less than or equal to 300 micrograms/mL in phosphate buffered saline (PBS) at pH=7.2-7.4. Examples include, but are not limited to, cyclosporin A, lovastatin, atorvastatin, dexamethasone, and travoprost isopropyl ester, latanoprost isopropyl ester. “Moderate solubility” means a solubility of greater than 300 micrograms/mL, but less than 1000 micrograms/mL in PBS at pH=7.2-7.4. Examples include, but are not limited to, latanoprost free acid (0.8 mg/mL in PBS), brimonidine tartrate (0.6 mg/mL in water at pH 7.7) and brimonidine free base (0.36 mg/mL in PBS). “High solubility” means a solubility of greater than or equal to 1000 micrograms/mL in PBS at pH=7.2-7.4. Examples include, but are not limited to, acetazolamide, dorzolamide HCl, timolol maleate, and ethacrynic acid sodium salt.

For ocular conditions, the active agent is suitably 3-hydroxy-2,2-bis(hydroxymethyl)propyl7-((1R,2R,3R,5S)-2-((R)-3-(benzo[b]thiophen-2-yl )-3-hydroxypropyl)-3,5-dihydroxycyclopentyl)heptanoate (AR-102), 7-((1R,2R,3R,5S)-2-((R)-3-(benzo[b]thiophen-2-yl)-3-hydroxypropyl)-3,5-dihydroxycyclopentyl)heptanoic acid (AR-102 free acid), dorzolamide, ethacrynic acid, latanoprost, latanoprost free acid, travoprost, travoprost free acid, bimatoprost, bimatoprost free acid, tafluprost, tafluprost free acid, dexamethasone, brimonidine, timolol, or salts thereof. Other suitable ocular active agents are known to those of ordinary skill in the art, such as other prostaglandins and other G-protein coupled receptor ligands, antifungals, antibiotics, enzyme inhibitors including kinase inhibitors, channel blockers, reuptake inhibitors and transporter inhibitors.

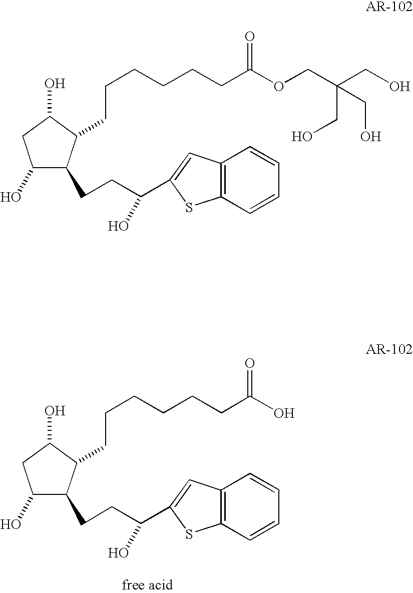

In some embodiments, the composition consists essentially of the active agent. In other embodiments, the composition also includes excipients such as the carriers and other components discussed below. The composition may be in the form of a single compressed pellet.

Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976). Examples of pharmaceutically acceptable carriers and excipients can, for example, be found in Remington Pharmaceutical Science, 16th Ed.

Suitable carriers include, but are not limited to, phosphate buffered saline (PBS), isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like.

The composition may also contain one or more of the following: a) diluents, b) binders, c) antioxidants, d) solvents, e) wetting agents, f) surfactants, g) emollients, h) humectants, i) thickeners, j) powders, k) sugars or sugar alcohols such as dextrans, particularly dextran 70, l) cellulose or a derivative thereof, m) a salt, and n) disodium EDTA (Edetate disodium).

Ingredient a) is a diluent. Suitable diluents for solid dosage forms include, but are not limited to sugars such as glucose, lactose, dextrose, and sucrose; diols such as propylene glycol; calcium carbonate; sodium carbonate; sugar alcohols, such as glycerin; mannitol; and sorbitol. The amount of diluent in the composition is typically about 0 to about 90%.

Ingredient b) is a binder. Suitable binders for solid dosage forms include, but are not limited to, polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder in the composition is typically about 0 to about 25%.

Ingredient c) is an antioxidant such as butylated hydroxyanisole (“BHA”), butylated hydroxytoluene (“BHT”), vitamin C and vitamin E. The amount of antioxidant in the composition is typically about 0 to about 15%.

Ingredient d) is a solvent such as water, ethyl alcohol, isopropanol, castor oil, ethylene glycol monoethyl ether, diethylene glycol monobutyl ether, diethylene glycol monoethyl ether, dimethylsulfoxide, dimethyl formamide, and combinations thereof. The amount of ingredient d) in the composition is typically about 0% to about 95%. While a solvent may be used, one discovery of the present invention is that a solvent is generally not needed to ensure substantially linear delivery of the active agent.

Ingredient e) is a wetting agent such as sodium lauryl sulfate, polyoxyethylene sorbitan fatty acid esters, polyoxyethylene alkyl ethers, sorbitan fatty acid esters, polyethylene glycols, polyoxyethylene castor oil derivatives, docusate sodium, quaternary ammonium compounds, sugar esters of fatty acids and glycerides of fatty acids.

Ingredient f) is a surfactant such as lecithin, Polysorbate 80, and sodium lauryl sulfate, and the TWEENS® from Atlas Powder Company of Wilmington, Del. Suitable surfactants include, but are not limited to, those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant in the composition is typically about 0% to about 5%.

Ingredient g) is an emollient. Suitable emollients include, but are not limited to, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. The amount of emollient in the composition is typically about 0% to about 50%.

Ingredient h) is a humectant. Suitable humectants include, but are not limited to, glycerin, sorbitol, sodium 2-pyrrolidone-5-carboxylate, soluble collagen, dibutyl phthalate, gelatin, and combinations thereof. The amount of humectant in the composition is typically about 0% to about 50%.

Ingredient i) is a thickener. The amount of thickener in the composition is typically about 0% to about 50%.

Ingredient j) is a powder. Suitable powders include, but are not limited to, beta-cyclodextrins, hydroxypropyl cyclodextrins, chalk, talc, fullers earth, kaolin, starch, gums, colloidal silicon dioxide, tetra alkyl ammonium smectites, trialkyl aryl ammonium smectites, chemically-modified magnesium aluminum silicate, organically-modified montmorillonite clay, hydrated aluminum silicate, fumed silica, sodium carboxymethyl cellulose, ethylene glycol monostearate, and combinations thereof. The amount of powder in the composition is typically about 0% to about 50%.

Ingredient m) is a cellulose derivative. Suitable cellulose derivatives include, but are not limited to, sodium carboxymethylcellulose, ethylcellulose, methylcellulose, and hydroxypropyl-methylcellulose, particularly, hydroxypropyl-methylcellulose.

Ingredient m) is a salt. Suitable salts include, but are not limited to, mono-, di- and trisodium phosphate, sodium chloride, potassium chloride, and combinations thereof.

The drug delivery devices of the present invention may be included in kits, which include the drug delivery devices and information, instructions, or both for use of the kit to provide treatment for medical conditions in mammals (particularly humans). The information and instructions may be in the form of words, pictures, or both, and the like.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The invention will be further explained by the following illustrative examples that are intended to be non-limiting.

Procedures for preparation of the drug delivery devices are described in the following examples. All temperatures are given in degrees Centigrade. Reagents were purchased from commercial sources (given) or prepared following literature procedures.

### Example 1

### Drug Delivery Device Containing Dorzolamide HCl (a High Solubility Drug)

### Parameters Tested

Thickness of permeable EVA film: 40-250 micrometers
Elution rate: 0.1-2 micrograms/hr

30 mg of dorzolamide HCl (which has high solubility) was compressed at 1000 psi to form a compressed drug pellet with a diameter of 5 mm and a thickness of 1 mm. Next, 15 mg of EVA-25 (vinyl acetate content of 25%; Sigma Chemical Company, St. Louis, Mo.) was loaded into a custom-made die set and heated to 100° C. for 1 minute. The polymer was compressed at 100 psi and allowed to cool to room temperature. When prepared in this manner, this EVA-25 polymer membrane is impermeable to water. The molded polymer cup was removed from the die set and the compressed drug pellet was loaded into the cup with the top side uncovered.

EVA-40 (Sigma Chemical Company, St. Louis, Mo.) was loaded into a film maker (International Crystal Laboratory) with a 150-micrometer spacer and heated to 75° C. for 4 minutes. The polymer was compressed at 1500 psi for 1 minute and allowed to cool to room temperature. The polymer membrane thus created with a thickness of 150 micrometers was removed from the base and cut into a disc-shaped membrane with a diameter of 6 mm using a biopsy punch. This polymer membrane is permeable to water when prepared in this manner. The disc-shaped, permeable membrane was placed on the exposed side of the drug pellet in contact with the EVA-25 “cup”, and the two polymers were heat-sealed at 90° C. using a custom-made die set and allowed to cool to room temperature.

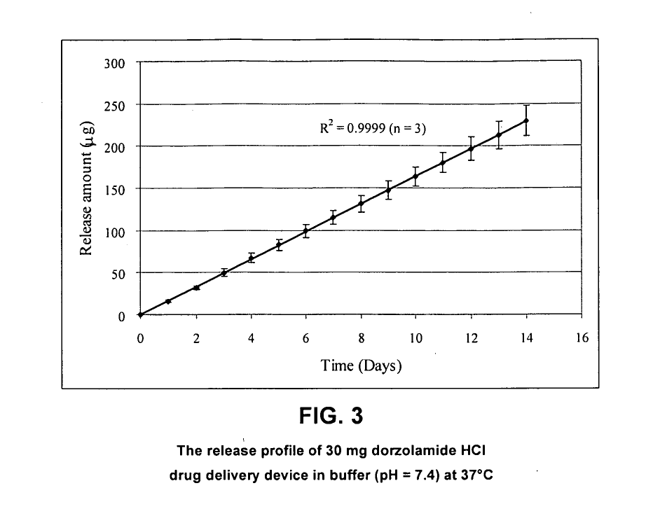

In summary, this drug delivery device was composed of a 30 mg core of dorzolamide HCl, the top and sides were composed of the impermeable EVA-25 polymer membrane, and the bottom of the drug delivery device was a 150 micrometer rate-limiting water-permeable membrane composed of EVA-40. The average elution rate in this particular design was 0.66±0.05 micrograms/hr (R²=0.9999) (FIG. 3).

### Example 2

### Drug Delivery Device Containing Ethacrynic Acid Sodium Salt (a High Solubility Drug)

### Parameters Tested

Thickness of EVA film: 100-500 micrometers
Elution rate: 5-50 micrograms/hr

30 mg of ethacrynic acid sodium salt (Sigma Chemical Company, St. Louis, Mo.) (which has high solubility), was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. When prepared in this manner, this polymer membrane was impermeable to water. The molded polymer cup was removed from the die set and the compressed drug pellet was loaded into the cup with the top side uncovered.

EVA-40 (Sigma Chemical Company, St. Louis, Mo.) was loaded into a film maker (International Crystal Laboratory) with a 25-micrometer spacer and heated to 75° C. for 4 minutes. The polymer was compressed at 200 psi for 1 minute and allowed to cool to room temperature. The thus created polymer membrane with a thickness of 75 micrometers was removed from the base and cut into a disc-shaped membrane with a diameter of 6 mm using a biopsy punch. This polymer membrane was permeable to water when prepared in this manner. The disc-shaped, permeable membrane was placed on the exposed side of the drug pellet in contact with the EVA-25 “cup”, and the two polymers were heat-sealed at 90° C. using a custom-made die set and allowed to cool to room temperature.

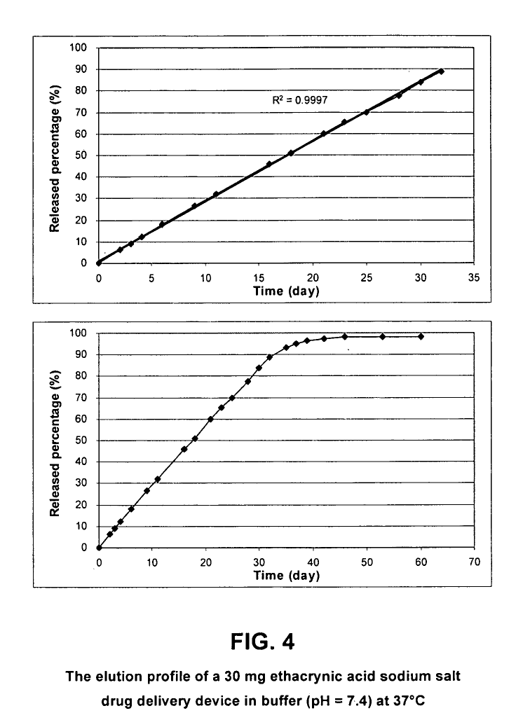

In summary, this drug delivery device was composed of a 30 mg core of ethacrynic acid sodium salt, the top and sides were composed of an impermeable EVA-25 polymer membrane, and the bottom of the drug delivery device was a 75 micrometer rate-limiting water-permeable membrane composed of EVA-40. The elution rate in this particular design was 27 micrograms/hr with a zero-order release profile for up to 90% of the contained agent (R²=0.9997) (FIG. 4).

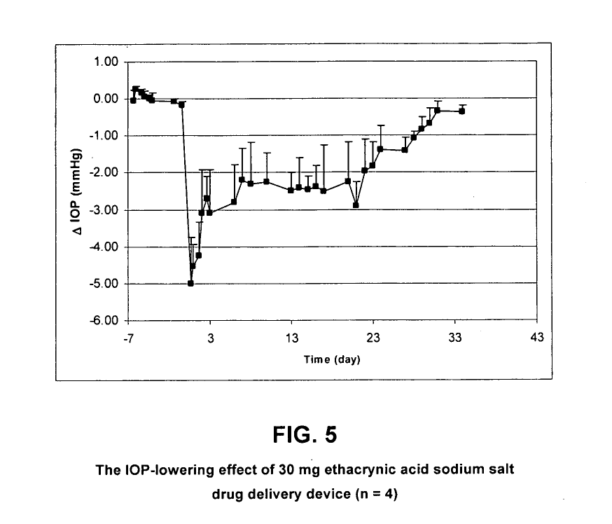

Ethacrynic acid sodium salt drug delivery devices falling within the above parameters with an elution rate of approximately 20 micrograms/hr were inserted episclerally in the right eye of Dutch-belted rabbits and the contralateral eye was used as an untreated control. The intraocular pressure was measured at regular intervals. As shown in FIG. 5, the devices provided a sustained IOP-lowering effect for approximately 30 days with >90% elution of the agent achieved.

### Example 3

### Drug Delivery Device Containing AR-102 Free Acid (a Moderately Soluble Drug)

### Parameters Tested

Thickness of EVA film: 120-250 micrometers
Elution rate: 0.04-0.7 micrograms/hr

4 mg of AR-102 free acid (which has moderate solubility) was compressed at 1000 psi to form a compressed drug pellet with a diameter of 3 mm and a thickness of 1 mm. 8 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

EVA-40 (Sigma Chemical Company, St. Louis, Mo.) was loaded into a film maker (International Crystal Laboratory) with a 200-micrometer spacer and heated to 75° C. for 4 minutes. The polymer was compressed at 200 psi for 1 minute and allowed to cool to room temperature. The polymer membrane with a thickness of 250 micrometers was removed from the base and cut into a disc-shaped membrane with a diameter of 4 mm using a biopsy punch. This polymer membrane was permeable to water when prepared in this manner. The disc-shaped, permeable membrane was placed on the exposed side of the drug pellet in contact with the EVA-25 “cup”, and the two polymers were heat-sealed at 90° C. using a custom-made die set and allowed to cool to room temperature.

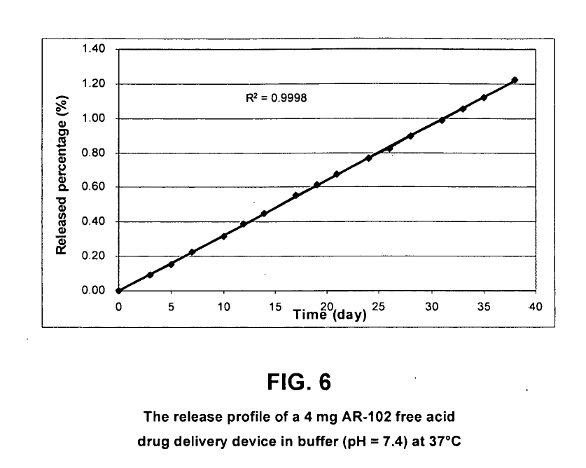

In summary, this device was composed of a 4 mg core of AR-102 free acid. The impermeable polymer was EVA-25. The rate-limiting water-permeable polymer was EVA-40, and the thickness of the water-permeable membrane was 250 micrometers. The elution rate in this particular design was 0.16 micrograms/hr (R²=0.9998) (FIG. 6).

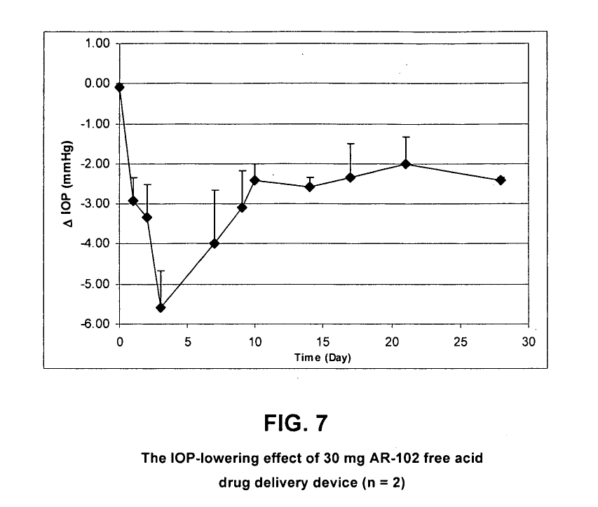

AR-102 free acid drug delivery devices falling within the above parameters with an elution rate of approximately 0.03 micrograms/hr were inserted episclerally in the right eye of Dutch-belted rabbits and the contralateral eye was used as an untreated control. The intraocular pressure was measured at regular intervals. As shown in FIG. 7, the devices provided a sustained IOP-lowering effect with a theoretical duration in vivo of approximately 7 years.

### Example 4

### Drug Delivery Device Containing Latanoprost Arginine Salt (a Moderately Soluble Drug)

### Parameters Tested

Thickness of EVA film: 40-300 micrometers
Elution rate: 0.04-5 micrograms/hr

4 mg of latanoprost arginine salt (which has moderate solubility) was compressed at 1000 psi to form a compressed drug pellet with a diameter of 3 mm and a thickness of 1 mm. 8 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

EVA-40 (Sigma Chemical Company, St. Louis, Mo.) was loaded into a film maker (International Crystal Laboratory) with a 150-micrometer spacer and heated to 75° C. for 4 minutes. The polymer was compressed at 400 psi for 1 minute and allowed to cool to room temperature. The polymer membrane with a thickness of 160 micrometers was removed from the base and cut into a disc-shaped membrane with a diameter of 4 mm using a biopsy punch. This polymer membrane was permeable to water when prepared in this manner. The disc-shaped, permeable membrane was placed on the exposed side of the drug pellet in contact with the EVA-25 “cup”, and the two polymers were heat-sealed at 90° C. using a custom-made die set and allowed to cool to room temperature.

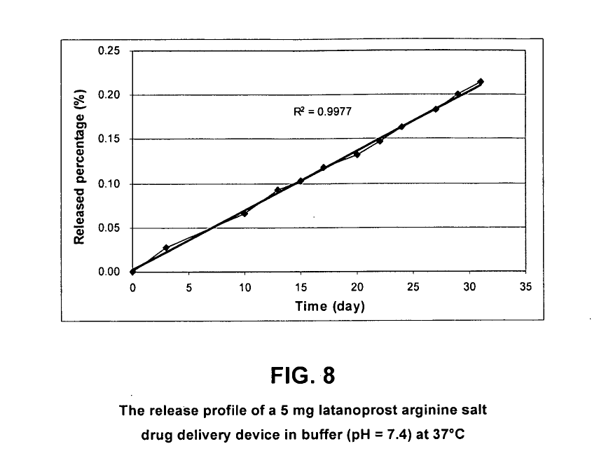

In summary, this device was composed of a 4 mg core of latanoprost arginine salt. The impermeable polymer was EVA-25. The rate-limiting water-permeable polymer was EVA-40, and the thickness of the water-permeable membrane was 160 micrometers. The elution rate in this particular design was approximately 0.01 micrograms/hr (R²=0.9977) (FIG. 8).

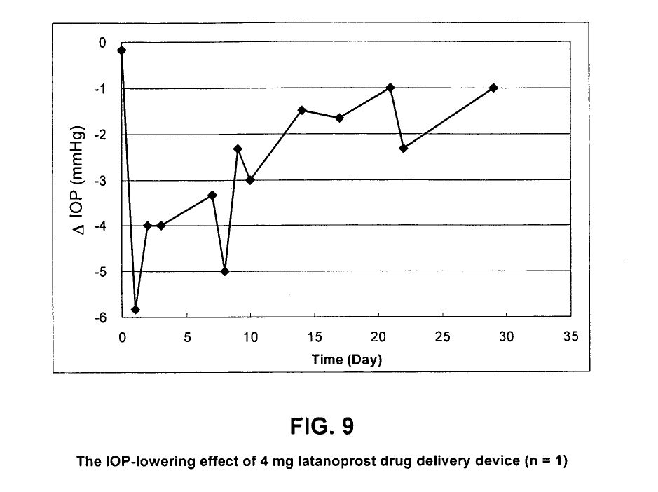

A latanoprost free acid arginine salt drug delivery device falling within the above parameters with an elution rate of approximately 0.01 micrograms/hr was inserted episclerally in the right eye of Dutch-belted rabbits and the contralateral eye was used as an untreated control. The intraocular pressure was measured at regular intervals. As shown in FIG. 9, the device provided a sustained IOP-lowering effect for approximately 30 days with a theoretical duration in vivo of approximately 30 years.

### Example 5

### Drug Delivery Device Containing Dexamethasone (a Low Solubility Drug)

### Parameters Tested

Thickness of EVA film: 40-150 micrometers
Elution rate: 0.1-3 micrograms/hr

30 mg of dexamethasone (which has low solubility) was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

EVA-40 (Sigma Chemical Company, St. Louis, Mo.) was loaded into a film maker (International Crystal Laboratory) with a 50-micrometer spacer and heated to 75° C. for 4 minutes. The polymer was compressed at 200 psi for 1 minute and allowed to cool to room temperature. The polymer membrane with a thickness of 75 micrometers was removed from the base and cut into a disc-shaped membrane with a diameter of 6 mm using a biopsy punch. This polymer membrane is permeable to water when prepared in this manner. The disc-shaped, permeable membrane was placed on the exposed side of the drug pellet, and the two polymers were heat-sealed at 90° C. using a custom-made die set and allowed to cool to room temperature.

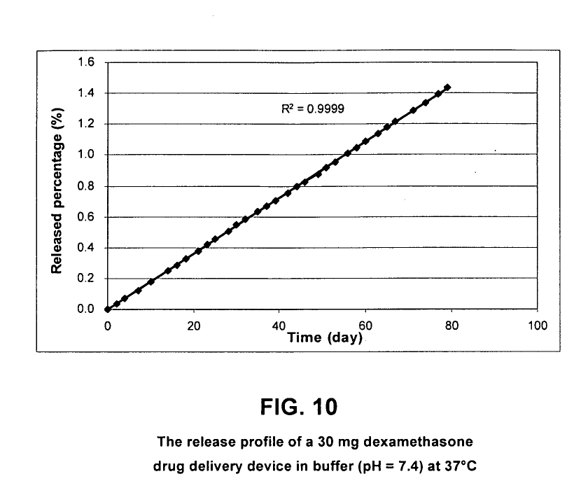

In summary, this device was composed of a 30 mg core of dexamethasone. The impermeable polymer was EVA-25. The rate-limiting water-permeable polymer was EVA-40, and the thickness of the water-permeable membrane was 75 micrometers. The elution rate in this particular design was 0.25 micrograms/hr (R²=0.9999) (FIG. 10).

### Example 6

### Ethylene Vinyl Acetate/Dextran Film

### Standard Methods for Making EVA/Dextran Film

Dextran with an average molecular weight of 5,000-670,000 Daltons (Fluka) was desiccated under vacuum overnight to purge excess moisture. EVA pellets with selected vinyl acetate ratios from 0 to 40% were ground into fine pieces to increase surface area. Dextran and EVA-0-40 were then measured out at a selected weight ratio in a sealed glass vial. Dichloromethane was incrementally added to the dextran/EVA mixture and the mixture was vigorously shaken to prevent clumping of dextran. The mixture was then gently heated to 50° C. and shaken in quick succession to aid EVA-25 dissolution. The mixture was then placed in an ultrasonic bath for 2 minutes. The mixture was allowed to cool to room temperature and inspected for undesirable air bubble formation.

A glass plate or silicon wafer was used as a casting substrate for the evaporative casting of the film. The mixture was uncapped and quickly decanted onto the substrate. Typical drying time was at least 4 hours under low humidity conditions to limit moisture uptake by the hygroscopic dextran. The cast film was then placed in a negative pressure rated flask and the atmosphere was flushed with high purity Argon gas. Air was then evacuated under a high vacuum overnight. The dried film was grounded into fine powder, and a dextran/EVA film with desired thickness was made by heat compression in a film maker. A digital micrometer was used to verify the final film thickness.

### Example 7

### Drug Delivery Device Containing Dexamethasone Sodium Phosphate (a High Solubility Drug)

### Parameters Tested

Dextran molecular weight: 5-12 kDa
Weight ratio of Dextran/EVA film: 1:20 to 1:4
Thickness of Dextran/EVA film: 40-150 micrometers
Elution rate: 2-200 micrograms/hr

30 mg of dexamethasone sodium phosphate (which has high solubility) was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

A mixture of EVA-25 (Sigma Chemical Company, St. Louis, Mo.) and dextran with an average molecular weight of 5 kDa was loaded into a film maker (International Crystal Laboratory) with a 100-micrometer spacer and heated to 100° C. for 4 minutes. The weight ratio of the dextran/EVA film was 1:19. The polymer was compressed at 200 psi for 1 minute and allowed to cool to room temperature. The polymer membrane with a thickness of 120 micrometers was removed from the base and cut into a disc-shaped membrane with a diameter of 6 mm using a biopsy punch. This was the partially-bioerodible membrane. The disc-shape, partially-bioerodible membrane was placed on the exposed side of the drug pellet in contact with the EVA-25 “cup”, and the two polymers were heat-sealed at 90° C. using a custom-made die set and allowed to cool to room temperature.

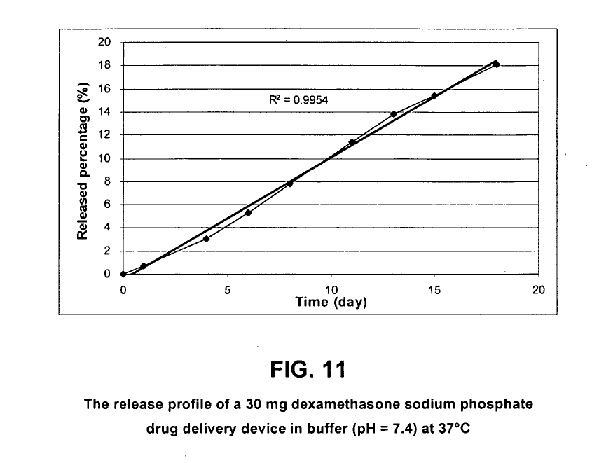

In summary, this device was composed of a 30 mg core of dexamethasone sodium phosphate. The impermeable polymer was EVA-25. The partially-bioerodible membrane was dextran with an average weight molecular of 5 kDa and EVA-25 at a weight ratio of 1:19, and the thickness of the partially-bioerodible membrane was 120 micrometers. The elution rate in this particular design was approximately 14 micrograms/hr (R²=0.9954) (FIG. 11).

### Example 8

### Drug Delivery Device Containing Brimonidine Free Base (a Low Solubility Drug)

### Parameters Tested

Dextran molecular weight: 12-670 kDa
Weight ratio of Dextran/EVA film: 1:4 to 1:3
Thickness of Dextran/EVA film: 40-150 micrometers
Elution rate: 0.05-4 micrograms/hr

20 mg of brimonidine free base (which has low solubility) was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

A mixture of EVA-25 (Sigma Chemical Company, St. Louis, Mo.) and dextran with an average molecular weight of 270 kDa was loaded into a film maker (International Crystal Laboratory) with a 50-micrometer spacer and heated to 75° C. for 4 minutes. The weight ratio of the dextran/EVA film was 1:4. The polymer was compressed at 400 psi for 1 minute and allowed to cool to room temperature. The polymer membrane which had a thickness of 65 micrometers was removed from the base and cut into a disc-shaped membrane with a diameter of 6 mm using a biopsy punch. This was the partially-bioerodible membrane. The disc-shaped, partially bioerodible membrane was placed on the exposed side of the drug pellet in contact with the EVA-25 “cup”, and the two polymers were heat-sealed at 90° C. using a custom-made die set and allowed to cool to room temperature.

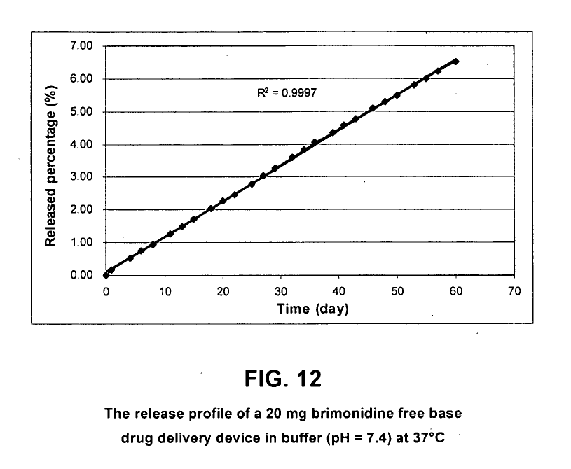

In summary, this device was composed of a 20 mg core of brimonidine free base. The impermeable polymer was EVA-25. The partially-bioerodible membrane was synthesized using dextran with an average molecular weight of 270 kDa and EVA-25 at a weight ratio of 1:4, and the thickness of the partially-bioerodible membrane was 65 micrometers. The elution rate in this particular design was 0.7 micrograms/hr (R²=0.9997) (FIG. 12).

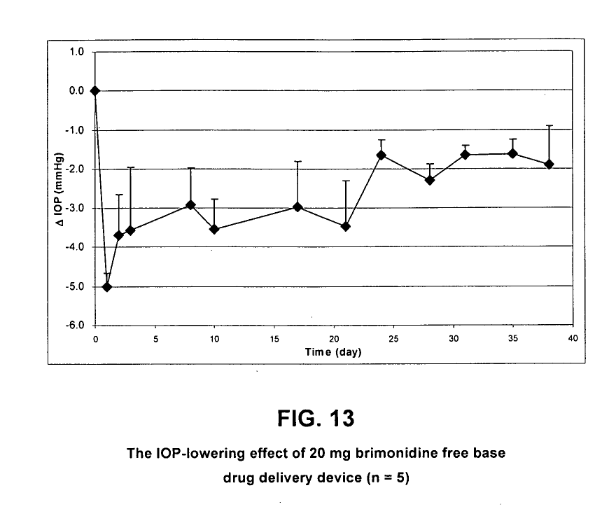

Brimonidine free base drug delivery devices falling within the above parameters using a similar design with elution rates of 0.7-2.5 micrograms/hr were inserted below the sclera in the right eye of Dutch-belted rabbits and the contralateral eye was used as an untreated control. The intraocular pressure was measured at regular intervals. As shown in FIG. 13, the device provided a sustained IOP-lowering effect for approximately 38 days with an expected duration in vivo of at least 7 months.

### Example 9

### Drug Delivery Device Containing Brimonidine D-Tartrate Salt (a High Solubility Drug)

### Parameters Tested

Dextran molecular weight: 5-270 kDa
Weight ratio of Dextran/EVA film: 1:20 to 1:4
Thickness of Dextran/EVA film: 95-150 micrometers
Elution rate: 0.3-60 micrograms/hr

30 mg of brimonidine D-tartrate salt (which has high solubility) was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

A mixture of EVA-25 (Sigma Chemical Company, St. Louis, Mo.) and dextran with an average molecular weight of 270 kDa was loaded into a film maker (International Crystal Laboratory) with a 100-micrometer spacer and heated to 100° C. for 4 minutes. The weight ratio of the dextran/EVA film was 1:4. The polymer was compressed at 200 psi for 1 minute and allowed to cool to room temperature. The polymer membrane which had a thickness of 125 micrometers was removed from the base and cut into a disc-shaped membrane with a diameter of 6 mm using a biopsy punch. This was the partially-bioerodible membrane. The disc-shaped, partially-bioerodible membrane was placed on the exposed side of the drug pellet in contact with the EVA-25 “cup”, and the two polymers were heat-sealed at 90° C. using a custom-made die set and allowed to cool to room temperature.

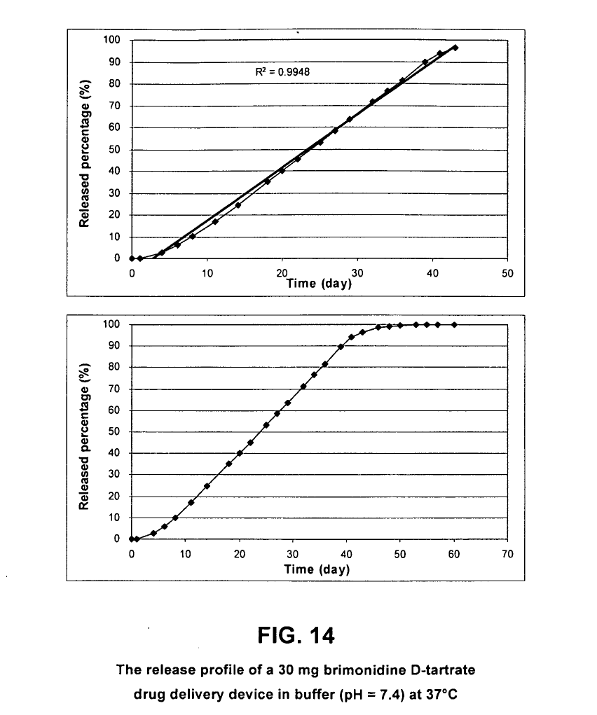

In summary, this device was composed of a 30 mg core of brimonidine D-tartrate salt. The impermeable polymer was EVA-25. The partially-bioerodible membrane was dextran with an average molecular weight of 270 kDa and EVA-25 at a weight ratio of 1:4, and the thickness of the partially-bioerodible membrane was 125 micrometers. The elution rate in this particular design was approximately 34 micrograms/hr with a zero-order release profile for up to 95% (R²=0.9948) (FIG. 14).

### Example 10

### Drug Delivery Device Containing Timolol Maleate Salt (a High Solubility Drug)

### Parameters Tested

Dextran molecular weight: 5-670 kDa
Weight ratio of Dextran/EVA film: 1:20 to 1:3
Thickness of Dextran/EVA film: 40-150 micrometers
Elution rate: 1-50 micrograms/hr

30 mg of timolol maleate (which has high solubility) was compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

A mixture of EVA-25 (Sigma Chemical Company, St. Louis, Mo.) and dextran with an average molecular weight of 5 kDa was loaded into a film maker (International Crystal Laboratory) with a 100-micrometer spacer and heated to 75° C. for 4 minutes. The weight ratio of the dextran/EVA film was 1:9. The polymer was compressed at 1500 psi for 1 minute and allowed to cool to room temperature. The polymer membrane which had a thickness of 100 micrometers was removed from the base and cut into a disc-shaped membrane with a diameter of 6 mm using a biopsy punch. This was the partially-bioerodible membrane. The disc-shape, partially-bioerodible membrane was placed on the exposed side of the drug pellet in contact with the EVA-25 “cup”, and the two polymers were heat-sealed at 90° C. using a custom-made die set and allowed to cool to room temperature.

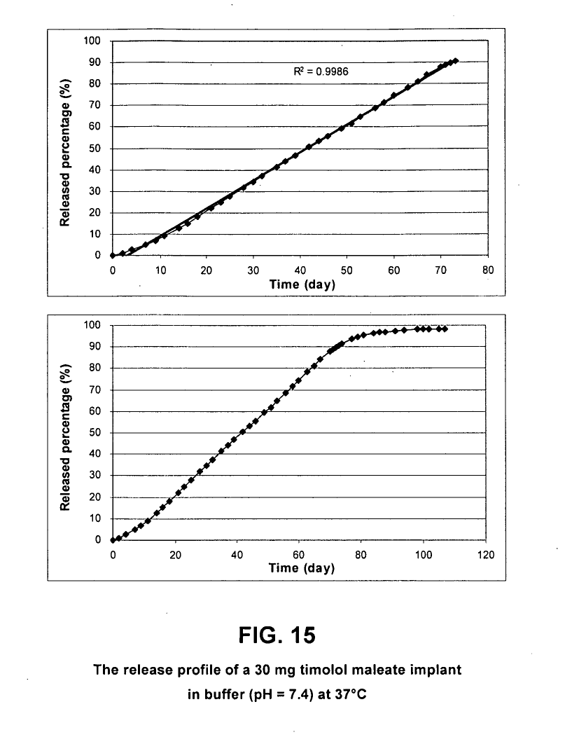

In summary, this device was composed of a 30 mg core of timolol maleate salt. The impermeable polymer was EVA-25. The partially-bioerodible membrane was dextran with an average molecular weight of 5 kDa and EVA-25 at a weight ratio of 1:9, and the thickness of the partially-bioerodible membrane was 100 micrometers. The elution rate in this particular design was approximately 15 micrograms/hr with a zero-order release profile for up to 90% of the enclosed agent (R²=0.9986) (FIG. 15).

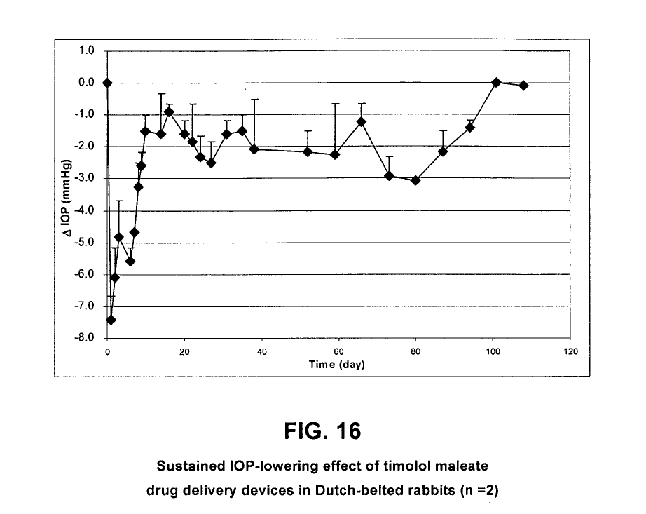

Timolol maleate salt drug delivery devices falling within the above parameters with elution rates of about 12 to 18 micrograms/hr were inserted below the sclera in the right eye of Dutch-belted rabbits and the contralateral eye was used as an untreated control. The intraocular pressure was measured at regular intervals. As shown in FIG. 16, the device provided a sustained IOP-lowering effect for approximately 90 days with complete elution achieved.

### Example 11

### Drug Delivery Device Containing Albumin (a High Molecular Weight, High Solubility Compound)

### Parameters Tested

Dextran molecular weight: 270-670 kDa
Weight ratio of Dextran/EVA film: 1:20 to 1:3
Thickness of Dextran/EVA film: 40-150 micrometers

30 mg of albumin (average molecular weight of approximately 67 kDa) that had been labeled with fluorescein isothiocyanate (BSA-FITC, Fluka) (which has high solubility) was mixed with unlabeled albumin at weight ratio of 1:9 and compressed at 1000 psi to form a compressed drug pellet with a diameter of 5 mm and a thickness of 1 mm. 15 mg of EVA-25 (Sigma Chemical Company, St. Louis, Mo.) was loaded into a custom-made die set and heated to 100° C. for 1 minute. The polymer was compressed at 100 psi and allowed to cool to room temperature. This was the impermeable polymer. The molded polymer cup was removed from the die set and the compressed drug pellet was loaded into the cup with the top side uncovered.

A mixture of EVA-25 (Sigma Chemical Company, St. Louis, Mo.) and dextran with an average molecular weight of 670 kDa was loaded into a film maker (International Crystal Laboratory) with a 50-micrometer spacer and heated to 100° C. for 4 minutes. The weight ratio of dextran/EVA film was 1:4. The polymer was compressed at 150 psi for 1 minute and allowed to cool to room temperature. The polymer membrane which had a thickness of 85 micrometers was removed from the base and cut into a disc-shaped membrane with a diameter of 6 mm using a biopsy punch. This was the partially-bioerodible membrane. The disc-shaped, partially-bioerodible membrane was placed on the exposed side of the drug pellet in contact with the EVA-25 “cup”, and the two polymers were heat-sealed at 90° C. using a custom-made die set and allowed to cool to room temperature.

In summary, this device was composed of a 30 mg core of albumin with 10% of the core consisting of FITC-labeled albumin. The impermeable polymer was EVA-25. The partially-bioerodible membrane was dextran with an average molecular weight of 670 kDa and EVA-25 at a weight ratio of 1:4, and the thickness of the partially-bioerodible membrane was 85 micrometers. The data showed that albumin was released from the permeable polymer at a controlled rate.

### Example 12

### General Methods of In Vitro Elution Rate Determination

A drug delivery device, containing a known active agent of interest, is placed in a 20-mL Class A clear borosilicate glass vial with PTFE threaded lid. To the vial is then added 10 mL of sterile 1× phosphate-buffered saline (PBS) without calcium and magnesium salts (Mediatech). The 20-mL glass vial is placed onto a tight fitting polymer rack. The polymer rack is then placed on an adjustable orbital platform shaker set to 60 Hz with infinite duration in a 37° C. incubator. At predetermined time points, 1-2 ml of the incubated solution is transferred from the vial to a sampling vial, and the rest of the solution is aspirated. The predetermined time intervals are usually 48 or 72 hours, and are subject to change based on the target elution rate and the maximum solubility of the active agent in PBS. 10 mL of fresh PBS is added to the 20-mL vial, and the vial is placed back to the incubator. In general, the concentration of active agent in solution is maintained at less than 10% of its maximum solubility in PBS to ensure the near-sink conditions.

The concentration of the solution in the sampling vial is determined using a standard curve obtained from several (usually more than 8) different known concentrations of the same active agent. The total amount of active agent eluted is determined from the original volume of the incubating solution and the elution rate is calculated based on the incubation time.

### Example 13

### Drug Delivery Device Containing Bimatoprost (a Low Solubility Drug)

### Suggested Parameters

Thickness of EVA film: 40-500 micrometers
Elution rate: 0.005-0.3 micrograms/hr
Preferred elution rate: 0.002-0.1 micrograms/hr

4 mg of bimatoprost (which has low solubility) is compressed at 1000 psi to form a compressed drug pellet with a diameter of 3 mm and a thickness of 1 mm. 8 mg of EVA-25 (Sigma) is loaded into a custom-made die set and heated to 100° C. for 1 minute. The polymer is compressed at 100 psi and allowed to cool to room temperature. This is the impermeable polymer. The molded polymer cup is removed from the die set and the compressed drug pellet is loaded into the cup with the top side uncovered.

EVA-40 is loaded into a film maker with a suitable spacer and heated to 75° C. for 4 minutes. The polymer is compressed at constant pressure for 1 minute and allowed to cool to room temperature. The polymer membrane with a thickness of 40-500 micrometers is removed from the base and cut into a disc-shaped membrane with a diameter of 4 mm using a biopsy punch. This polymer membrane is permeable to water when prepared in this manner. The disc-shaped, permeable membrane is placed on the exposed side of the drug pellet in contact with the EVA-25 “cup”, and the two polymers are heat-sealed at 90° C. using a custom-made die set and allowed to cool to room temperature.

In summary, this device is composed of a 4 mg core of bimatoprost. The top and sides are composed of an impermeable EVA-25 polymer membrane, and the bottom of the drug delivery device is a 40-500 micrometer permeable membrane composed of EVA-40. The elution rate in this design can be adjusted to the desired elution rate by changing the thickness of the permeable polymer.

### Example 14

### Drug Delivery Device Containing Latanoprost Isopropyl Ester (a Low Solubility Drug)

### Suggested Parameters

Thickness of EVA film: 300-1000 micrometers Elution rate: 0.005-0.3 micrograms/hr
Preferred elution rate: 0.001-0.05 micrograms/hr

8 mg of EVA-25 is loaded into a custom-made die set and heated to 100° C. for 1 minute. The polymer is compressed at 100 psi and allowed to cool to room temperature. This is the impermeable polymer. The molded polymer cup is removed from the die set and 4 mg of latanoprost isopropyl ester (which has low solubility) is loaded into the EVA-25 cup.

EVA-40 is loaded into a film maker with a suitable spacer and heated to 75° C. for 4 minutes. The polymer is compressed at constant pressure for 1 minute and allowed to cool to room temperature. The polymer membrane with a thickness of 300-800 micrometers is removed from the base and cut into a disc-shaped membrane with a diameter of 4 mm using a biopsy punch. This polymer membrane is permeable to water when prepared in this manner. The disc-shaped, permeable membrane is placed on the exposed side of the drug pellet in contact with the EVA-25 “cup”, and the two polymers are heat-sealed at 90° C. using a custom-made die set and allowed to cool to room temperature.

In summary, this device is composed of a 4 mg core of latanoprost isopropyl ester. The top and sides are composed of an impermeable EVA-25 polymer membrane, and the bottom of the drug delivery device is a 40-500 micrometer permeable membrane composed of EVA-40. The elution rate in this design can be adjusted to desired elution rate by changing the thickness of the permeable polymer.

### Example 15

### Drug Delivery Device Containing Travoprost Isopropyl Ester (a Low Solubility Drug)

### Suggested Parameters

Thickness of EVA film: 300-750 micrometers
Elution rate: 0.001-0.04 micrograms/hr
Preferred elution rate: 0.001-0.02 micrograms/hr

8 mg of EVA-25 is loaded into a custom-made die set and heated to 100° C. for 1 minute. The polymer is compressed at 100 psi and allowed to cool to room temperature. This is the impermeable polymer. The molded polymer cup is removed from the die set and 4 mg of travoprost isopropyl ester (which has low solubility) is loaded into the EVA-25 cup.

EVA-40 is loaded into a film maker (International Crystal Laboratory) with a suitable spacer and heated to 75° C. for 4 minutes. The polymer is compressed at constant pressure for 1 minute and allowed to cool to room temperature. The polymer membrane with a thickness of 300-800 micrometers is removed from the base and cut into a disc-shaped membrane with a diameter of 4 mm using a biopsy punch. This polymer membrane is permeable to water when prepared in this manner. The disc-shaped, permeable membrane is placed on the exposed side of the drug pellet in contact with the EVA-25 “cup”, and the two polymers are heat-sealed at 90° C. using a custom-made die set and allowed to cool to room temperature.

In summary, this device is composed of a 4 mg core of travoprost isopropyl ester. The top and sides are composed of an impermeable EVA-25 polymer membrane, and the bottom of the drug delivery device is a 40-500 micrometer permeable membrane composed of EVA-40.

### Example 16

### Drug Delivery Device Containing Non-steroidal Anti-Inflammatory Drugs

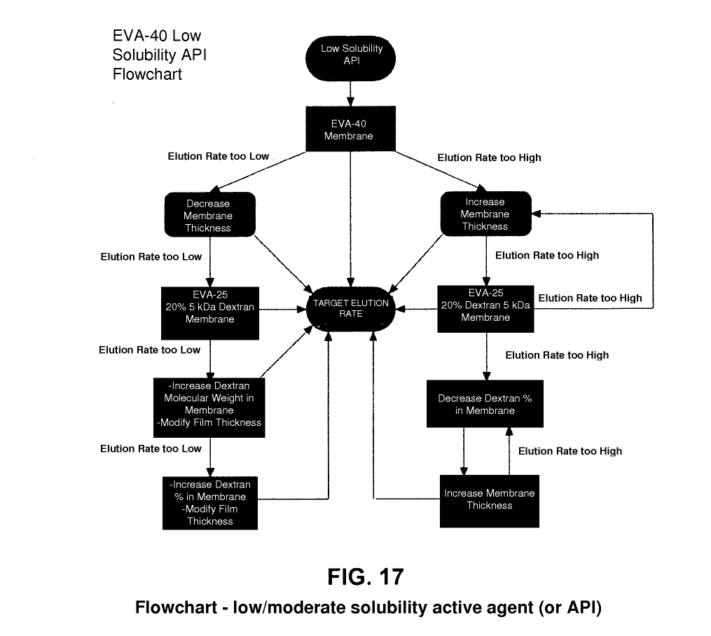

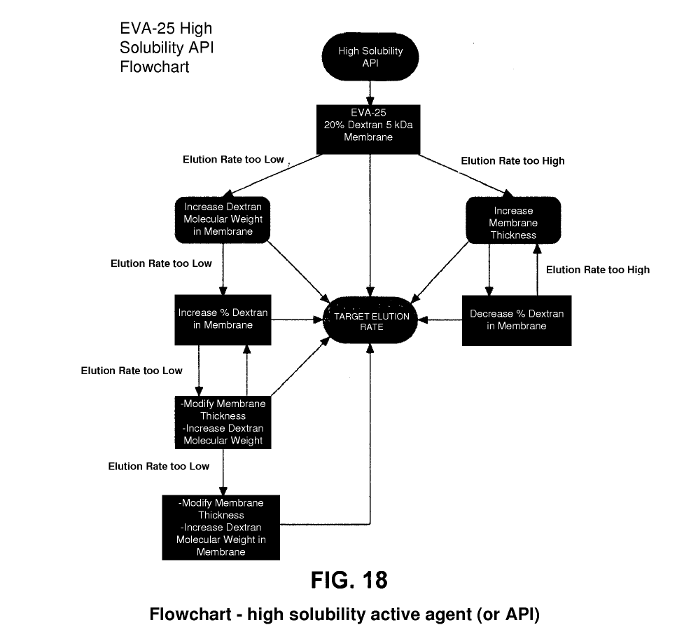

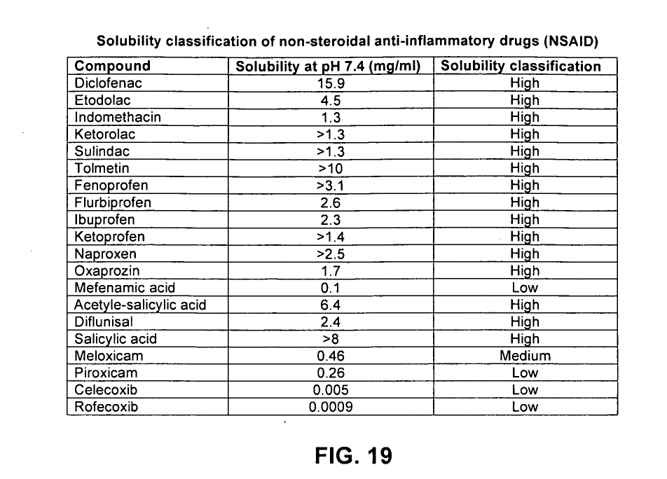

A drug delivery device of the invention can be designed to release a selected active agent at a predetermined rate using the flowcharts and table in FIGS. 17-19. Suitably, one would start with EVA-40 as the water permeable membrane and EVA-25 as the water impermeable membrane, or using partially-bioerodible membranes if the active agent may not release at the predetermined rate. For those skilled in the art, the composition and thickness of the membrane can readily be identified using similar experimental procedures illustrated above.

1. A drug delivery device comprising:
a composition comprising an active agent; and
a non-bioabsorbable polymer structure enclosing the composition, the polymer structure comprising a mixture comprising a water-soluble polymer and a non-water-soluble polymer.

2. The drug delivery device of claim 1, wherein the device at least partially bioerodes when implanted in a body as the water-soluble polymers dissolve leaving a porous structure, through which the active agent is released.

3. The drug delivery device of claim 1, wherein the active agent is an ocular active agent.

4. The drug delivery device of claim 1, wherein the active agent is selected from the group consisting of AR-102, AR-102 free acid, dorzolamide HCl, ethacrynic acid, latanoprost, latanoprost free acid, travoprost, travoprost free acid, bimatoprost, bimatoprost free acid, tafluprost, tafluprost free acid, dexamethasone, brimonidine, timolol, and salts thereof.

5. The drug delivery device of claim 1, wherein the non-water-soluble polymer is selected from the group consisting of EVA-9-50, silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutymethacrylate, plasticized polyethyleneterephthalate, polyisoprene, polyisobutylene, silicon-carbon copolymers, natural rubber, plasticized soft nylon, and polytetrafluoroethylene (PTFE).

6. The drug delivery device of claim 1, wherein the water-soluble polymer is selected from the group consisting of dextran, cyclodextrin, poly-(L-lactic acid), polycaprolactone, poly(lactic-co-glycolic acid), poly(glycolic acid), poly(trimethylene carbonate), and polydioxanone.

7. The drug delivery device of claim 1, wherein the polymer structure has a thickness of about 20 micrometers to about 800 micrometers.

8. The drug delivery device of claim 1, wherein the active agent is released from the drug delivery device at rate of about 0.0001 to about 30 micrograms/hr.

9. The drug delivery device of claim 1, wherein the rate of release of active agent does not substantially deviate from linearity until at least about 70% and at most about 95% of active agent is released from the drug delivery device.

10. The drug delivery device of claim 1, wherein about 70% to about 90% of the active agent is released from the drug delivery device within about 30 days to about 5 years.

11. A drug delivery device comprising:
a composition comprising an active agent; and
a non-bioabsorbable polymer structure enclosing the composition, the polymer structure comprising an impermeable polymer through which the active agent does not permeate and a partially-bioerodible membrane through which the active agent permeates.

12. The drug delivery device of claim 11, wherein the active agent is an ocular active agent.

13. The drug delivery device of claim 11, wherein the active agent is selected from the group consisting of AR-102, AR-102 free acid, dorzolamide, ethacrynic acid, latanoprost, latanoprost free acid, travoprost, travoprost free acid, bimatoprost, bimatoprost free acid, tafluprost, tafluprost free acid, dexamethasone, brimonidine, timolol, and salts thereof.

14. The drug delivery device of claim 11, wherein the impermeable polymer is selected from the group consisting of EVA-9-50, silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutylmethacrylate, plasticized polyethyleneterephthalate, polyisoprene, polyisobutylene, silicon-carbon copolymers, natural rubber, plasticized soft nylon, and polytetrafluoroethylene (PTFE).

15. The drug delivery device of claim 11, wherein the partially-bioerodible membrane comprises a mixture comprising impermeable polymer and a bioerodible polymer.

16. The drug delivery device of claim 15, wherein the bioerodible polymer is selected from the group consisting of dextran, cyclodextrin, poly-(L-lactic acid), polycaprolactone, poly(lactic-co-glycolic acid), poly(glycolic acid), poly(trimethylene carbonate), and polydioxanone.

17. The drug delivery device of claim 11, wherein the impermeable polymer has a thickness of about 50 micrometers to about 800 micrometers.

18. The drug delivery device of claim 11, wherein the partially-bioerodible membrane has a thickness of about 20 micrometers to about 800 micrometers.

19. The drug delivery device of claim 11, wherein the drug delivery device has a cylindrical structure comprising a cylindrical wall comprising the impermeable polymer and a top comprising the impermeable polymer coupled to the cylindrical wall, and a bottom comprising the partially-bioerodible membrane coupled to the cylindrical wall.

20. The drug delivery device of claim 11, wherein the active agent is released from the drug delivery device at rate of about 0.0001 to about 30 micrograms/hr.

21. The drug delivery device of claim 11, wherein the rate of release of active agent does not substantially deviate from linearity until at least about 70% and at most about 95% of active agent is released from the drug delivery device.

22. The drug delivery device of claim 11, wherein about 70 to about 90% of the active agent is released from the drug delivery device within about 30 days to about 5 years.

23. A drug delivery device comprising:
a composition comprising a single compressed pellet comprising an active agent with a solubility of greater than about 50 micrograms/mL in phosphate buffered saline at a neutral pH; and
a non-bioabsorbable polymer structure enclosing the composition, the polymer structure comprising an impermeable polymer through which the active agent does not permeate and a rate-limiting water-permeable polymer through which the active agent permeates.

24. The drug delivery device of claim 23, wherein the active agent is an ocular active agent.

25. The drug delivery device of claim 23, wherein the active agent is selected from the group consisting of AR-102, AR-102 free acid, dorzolamide, ethacrynic acid, latanoprost, latanoprost free acid, travoprost, travoprost free acid, bimatoprost, bimatoprost free acid, tafluprost, tafluprost free acid dexamethasone, brimonidine, timolol, and salts thereof.

26. The drug delivery device of claim 23, wherein the impermeable polymer is selected from the group consisting of EVA-9-50, silicon rubber polymers, polydimethylsiloxane (PDMS), polyurethane (PU), polyesterurethanes, polyetherurethanes, polyolefins, polyethylenes (PE), low density polyethylene (LDPE), polypropylene (PP), polyetheretherketone (PEEK), polysulfone (PSF), polyphenylsulfone, polyacetals, polymethyl methacrylate (PMMA), polybutylmethacrylate, plasticized polyethyleneterephthalate, polyisoprene, polyisobutylene, silicon-carbon copolymers, natural rubber, plasticized soft nylon, and polytetrafluoroethylene (PTFE).

27. The drug delivery device of claim 23, wherein the water-permeable polymer is selected from the group consisting of EVA 35-80 and ethylene vinyl alcohol 9-80.

28. The drug delivery device of claim 23, wherein the water-impermeable polymer has a thickness of about 50 micrometers to about 800 micrometers.

29. The drug delivery device of claim 23, wherein the water-permeable polymer has a thickness of about 20 micrometers to about 500 micrometers.

30. The drug delivery device of claim 23, wherein the drug delivery device has a cylindrical structure comprising a cylindrical wall comprising the impermeable polymer and a top comprising the impermeable polymer coupled to the cylindrical wall, and a bottom comprising the partially-bioerodible membrane coupled to the cylindrical wall.

31. The drug delivery device of claim 23, wherein about 5% to about 80% of the active agent is released from the drug delivery device with an R² of at least about 0.95.

32. The drug delivery device of claim 23, wherein the active agent is released from the drug delivery device at rate of about 0.0001 to about 30 micrograms/hr.

33. The drug delivery device of claim 23, wherein the rate of release of active agent does not substantially deviate from linearity until at least about 70% and at most about 95% of active agent is released from the drug delivery device.

34. The drug delivery device of claim 23, wherein about 70 to about 90% of the active agent is released from the drug delivery device within about 30 days to 5 years.

35. A drug delivery device comprising a composition comprising an active agent at least partially encompassed by an impermeable membrane and a permeable membrane, wherein the permeable membrane controls release of the active agent episclerally over a period of time.

36. The drug delivery device of claim 35, wherein the active agent is an ocular active agent.

37. The drug delivery device of claim 35, wherein the active agent is selected from the group consisting of AR-102, AR-102 free acid, dorzolamide, ethacrynic acid, latanoprost, latanoprost free acid, travoprost, travoprost free acid, bimatoprost, bimatoprost free acid, tafluprost, tafluprost free acid, dexamethasone, brimonidine, timolol, and salts thereof.

38. A method of treating an ocular condition comprising implanting the drug delivery device of claim 1 at or near a tissue affected by the condition.

39. The method of claim 38, wherein the ocular condition is ocular hypertension or glaucoma.

40. The method of claim 38, wherein the drug delivery device is implanted episclerally.

41. A method of treating an ocular condition comprising implanting the drug delivery device of claim 11 at or near a tissue affected by the condition.

42. The method of claim 41, wherein the ocular condition is ocular hypertension or glaucoma.

43. The method of claim 41, wherein the drug delivery device is implanted episclerally.

44. A method of treating an ocular condition comprising implanting the drug delivery device of claim 23 at or near a tissue affected by the condition.

45. The method of claim 44, wherein the ocular condition is ocular hypertension or glaucoma.

46. The method of claim 44, wherein the drug delivery device is implanted episclerally.

47. A method of treating an ocular condition comprising implanting episclerally a drug delivery device comprising an active agent, wherein the active agent is released at a rate of
Q=0.001×N×C
wherein C is the topical effective concentration (in milligram/mL) of the active agent and N=0.01 to 0.5 for prostaglandins in their ester, amide, free acid or salt form, and N=0.5 to 5 for any active agent other than prostaglandins in their ester, amide, free acid or salt form.
